(19) United States
(12) Patent Application Publication
(10) Pub. No.: US 2008/0146969 A1
(43) Pub. Date: Jun. 19, 2008

(54) Total joint replacement component positioning as predetermined distance from center of rotation of the joint using pinless navigation

(76) Inventor: William B. Kurtz, Nashville, TN (US)

Correspondence Address: Kenneth A. Roddy, Suite 100, 2916 West T.C. Jester, Houston, TX, 77018, US

(21) Appl. No.: 11/975,515

(22) Filed: Oct. 19, 2007

Related U.S. Application Data
(63) Continuation-in-part of application No. 11/640,141, filed on Dec. 15, 2006.

(51) Int. Cl. A61B 5/103; A61B 17/58

(52) U.S. Cl. 600/595; 606/102

(57) ABSTRACT

A system and method used in total joint arthroplasty of a ball and socket joint such as hip and shoulder replacements for accurate positioning of a prosthesis through pinless surgical navigation during replacement surgery according to a predetermined distance from the center of rotation of the replaced joint and/or articular surface to obtain the proper length, offset, and biomechanics of the replaced joint.

### CROSS REFERENCE TO RELATED APPLICATION

This application is a Continuation-In-Part of, and claims the benefit of priority under 35 U.S.C. 120, of copending U.S. patent application Ser. No. 11/640,141, filed Dec. 15, 2006, and which is expressly incorporated herein by reference in its entirety.

### BACKGROUND OF THE INVENTION

1. Field of the Invention

This invention relates generally to total joint arthroplasty of a ball and socket joint such as hip and shoulder replacements, and more particularly to a system and method for accurate positioning of a prosthesis through pinless surgical navigation during replacement surgery according to a predetermined distance from the center of rotation of the replaced joint and/or articular surface in order to obtain the proper length, offset, and biomechanics of the replaced joint.

2. Background Art

After hip replacement surgery, patients often suffer from leg length displacements, lateral offset displacements, or a translation of the center of rotation of the hip. Often, these patients suffer from a persistent limp, an abnormal gait, and inappropriate soft tissue tension after hip replacement surgery. These complications are normally the result of an inability to properly reposition the femur in relation to the ilium as a result of improper positioning or selection of the femoral or acetabular component. This inability is normally caused by the lack of measurements of displacements caused by the hip replacement when the femur is reconnected to the ilium.

Unfortunately, current methods of relocating the hip are unacceptably inaccurate or are relatively bulky and complicated to install and use. Prior art methods determine the leg length and lateral offset displacements by placing reference points on the femur and the patient's ilium. Inserted into the ilium reference points are reference pins for measuring the relative displacement of the patient's femur to the selected points on the ilium. Typically, the reference point on the femur is located on the greater trochanter since it is on the upper part of the femur and hence relatively close to the patient's ilium so as to facilitate measurement. The ilium reference pins extrude through the skin and are normally reached through a stab incision above the acetabulum.

Most prior art measurement devices suffer from several disadvantages. First, prior art devices do not measure relative to the femoral axis of the femur. Therefore, rotations of the femur during surgery can lead to inaccuracies in the measurement of displacements. Second, measurement error exists when the reference pins loosen from the bone and there is relative motion between the bones and the reference pins. Furthermore, these reference pins are placed at an unacceptable distance from the hip causing them to bend and rotate throughout the surgery. The prior art thus provides unstable reference locations for measuring the displacements of the femur. Third, the reference pins are not removable and thus obstruct the surgeon during portions of the surgery. Fourth, these pins require extra incisions including a blind insertion of the pin into the ilium which risks neurovascular injury. Finally, many of these prior art devices introduce error because the leg is not returned to its original angular orientation (i.e. rotation, flexion, abduction) for displacement measurements.

My copending U.S. patent application Ser. No. 11/640,141, which the present application is a C-I-P of, and which is expressly incorporated herein by reference in its entirety, discloses a joint measurement device and method for determining length displacement and lateral offset displacement of a patient's femur during hip replacement surgery. The device and method permit the surgeon to measure the leg length displacement and the lateral offset displacement in order to select components for a replacement hip that returns the femur to its original position. The device may also be utilized to maintain the hip's center of rotation and native anteversion. More particularly, the device and method improves the spatial measurement of the relationship between a femur and ilium prior to the hip replacement and can accurately recreate that spatial relationship after the hip replacement surgery. The device and method stabilizes the reference points between the femur and ilium and provides a more accurate measurement of displacements between the femur and ilium thereby facilitating a proper selection of the hip replacement and providing the appropriate alignment between the femur and ilium.

Various surgical navigation procedures for hip arthroplasty have been developed and continue to improve. To date, some total hip arthroplasty surgical navigation systems attach active or passive line-of-sight navigation arrays, tracking sensors, or radio frequency markers to both the ilium and femoral bone. Measurements of distances between these devices both before and after the hip replacement help surgeons recreate leg length and offset.

Surgeons attempt to determine the depth at which the femoral broach and/or prosthesis is likely to become firmly attached to the femoral bone by using pre-operative x-rays and transparent femoral prosthetic templating guides. By selecting a smaller or larger sized femoral prosthetic templating guide, the surgeon can increase or decrease the planned depth of the femoral prosthesis so that the known femoral neck length corresponds with the planned distance between the femoral and acetabular prosthesis. This planned depth of the femoral component may or may not correspond with the actual depth that is obtained during surgery due to errors in radiograph magnification, leg positioning, and inaccurate templating. Distances from the planned depth of the femoral prosthesis and known bony landmarks (i.e. lesser troachanter) on the femoral bone are measured on the pre-operative x-rays, and the surgeon attempts to reproduce those measurements during surgery in hopes to increase his/her accuracy in leg length and offset.

There are several patents directed toward various “pinless” surgical navigation devices and procedures used in ball and socket joint replacement surgery, such as hip and shoulder arthroplasty.

Sarin, et al, U.S. Pat. No. 6,711,431 and U.S. Published Patent Application 20040254584 disclose a non-imaging, computer assisted navigation system for hip replacement surgery, which includes: a locating system; a computer, interfaced to the locating system and interpreting the positions of tracked objects in a generic computer model of a patient's hip geometry; a software module, executable on the computer, which defines the patient's pelvic plane without reference to previously obtained radiological data, by locating at least three pelvic landmarks; and a pelvic tracking marker, fixable to the pelvic bone and trackable by the locating system, to track in real time the orientation of the defined pelvic plane. Preferably, the system also includes a femoral tracking marker, securely attachable to a femur of the patient by a non-penetrating ligature and trackable by the locating system to detect changes in leg length and femoral offset.

Jansen, et al, U.S. Published Patent Application 20040230199 discloses a computer-assisted surgery (CAS) system and method for guiding an operator in inserting a femoral implant in a femur as a function of a limb length and orientation of the femoral implant with respect to the femur, comprising a reference tool for the femur, a registration tool, a bone altering tool and a sensing apparatus. A controller is connected to the sensing apparatus to: (i) register a frame of reference of the femur by calculating surface information provided by the registration tool as a function of the position and orientation of the registration tool provided by the sensing apparatus, and/or retrieving in a database a model of the femur; (ii) calculate a desired implant position with respect to the frame of reference as a function of the limb length; and (iii) calculate a current implant position and orientation in relation to the desired implant position with respect to alterations being performed in the femur with the bone altering tool, as a function of the position and orientation of the bone altering tool provided by the sensing apparatus and of a digital model of a femoral implant provided by the database. The database is connected to the controller for the controller to store and retrieve information relating to an operation of the controller. The computer-assisted system may be used to guide an operator in inserting a pelvic implant in an acetabulum as a function of an orientation of the pelvic implant with respect to the pelvis.

Murphy, U.S. Pat. No. 7,105,028 (was 20050081867) discloses a minimally invasive and tissue preserving surgical procedure for the replacement of a hip joint, including the steps of: making a superiorly positioned incision; and preparing the femoral canal of a patient's natural femur for receipt of a femoral implant, through the superior incision, while the patient's natural femoral head is still within the patient's natural acetabulum.

Radinsky, et al, U.S. Published Patent Application 20060293614 discloses a computer-assisted surgery (CAS) system and method for measuring surgical parameters during hip replacement surgery to guide an operator in inserting a hip joint implant in a femur, comprising a first trackable reference in fixed relation with the pelvis and a registration tool. A sensor apparatus tracks the first trackable reference and the registration tool. A controller unit is connected to the sensor apparatus so as to receive tracking data for the first trackable reference and the registration tool. The controller unit has a position and orientation calculator to calculate from the tracking data a position and orientation of the pelvic trackable reference to track the pelvic frame of reference, and of the registration tool to produce a femoral frame of reference at two sequential operative steps. A reference orientation adjustor receives tracking data for the pelvic frame of reference, and the femoral frame of reference associated with the first trackable reference, to orient the femoral frame of reference in a reference orientation with respect to the pelvic frame of reference, and to produce a reference adjustment value as a function of the reference orientation. A surgical parameter calculator receives tracking data from the registration tool to calculate surgical parameters as a function of the reference adjustment value, the surgical parameters at the two sequential operative steps being related by the reference orientation.

Amiot, et al, U.S. Published Patent Application 20060287613 discloses a device, method and system for digitizing a center of rotation of a hip component with respect to a bone element in computer-assisted surgery. The device comprises a detectable member trackable for position and orientation by a computer-assisted surgery system. A body is connected to the detectable member in a known geometry. The body has a coupling portion adapted to be coupled to the hip joint implant component in a predetermined configuration. In one embodiment, the coupling of the detectable member is a tubular body having a cylindrical bore that is received on the frusto-conically shaped connector end of the femoral implant. In an alternate embodiment, the connector has a hemispherical hole and is positioned directly on the ball head secured to the connector end of the femoral implant. A detector member is also disclosed for digitizing the center of rotation of the acetabular implant, which has a generally hemispherical body that is positioned into the receiving cavity of the shell of the acetabular implant. The center of rotation of the hip component is calculable in the predetermined configuration as a function of the known geometry and of the position and orientation of the detectable member.

It should be noted that the present invention differs from Amoit, et al, in that the present method determines the center of rotation of the actual ball and socket hip joint, whereas Amoit, et al, determines the center of rotation of the prosthesis (which could also just be determined from a data-base of known implants.

Murphy, U.S. Published Patent Application 20060264731 discloses methods of determining the axial rotation and transaxial rotation of a pelvis from a single fluoroscopic image, comprising the steps of: (A) forming a fluoroscopic image of said pelvis in the near AP direction; (B) defining first and second landmarks of the pelvis on the image, the landmarks separated from each other in at least an anterior-posterior direction; (C) determining the axial and transaxial displacement of the landmarks on the image; and (D) using the displacement as a measure of the axial and transactional rotation of the pelvis with respect to the plane of the fluoroscopic image.

Moctezuma de la Barrera, et al, U.S. Published Patent Application 20050065617 discloses a system and method of performing a total replacement surgery of a ball and socket joint of a patient using a surgical navigation system that is performed by constructing intra-operatively a three dimensional model of the joint based on landmarks of the patient, by preparing the joint to receive implants, by placement of implants into the prepared joint and by determining range of motion and/or stability of the reconstructed joint. The system includes a surgical navigation system, a first circuit to construct intra-operatively a three dimensional model of the joint, a first tool to prepare the joint, a second tool to place an implant into the prepared joint, and a second circuit to determine range of motion and/or stability of the reconstructed joint. A virtual trialing or look ahead feature can also be included. A tool to locate the center of the canal of a limb includes an elongate body, a series of outwardly biased surfaces spaced around the elongate body and an interface to enable a tracking device to be attached to the body. A tool to guide the depth of the resection of a neck of a limb comprises a flat guide surface, a handle, and an interface to enable a tracking device to be attached to the tool.

Another problem with current and prior art ball and socket joint replacement surgery procedures, such as hip and shoulder arthroplasty, is that surgeons typically cut the neck of the femoral or humeral prosthesis and then inserted broaches into the femoral or humeral canal. The surgeon gradually enlarges the size of the broach until the broach fills the canal and stops at a pre-determined point. The surgeon typically measures on a pre-operative x-ray where he/she wants the broach to sit in the femoral or humeral canal so he/she can recreate the limb length and offset. The surgeon typically estimates the location of the broach to a pre-determined bony reference point (lesser trochanter) and compares the intra-operative measurement with the measurement on the pre-operative x-ray. The surgeon can then increase the size of the femoral or humeral broach until it is firmly positioned in the proper location (as best as the surgeon can tell).

### SUMMARY OF THE INVENTION

It is therefore an object of the present invention to provide a surgical navigation method that accurately positions a prosthesis through pinless surgical navigation during replacement surgery according to a predetermined distance from the center of rotation of the replaced joint and/or articular surface to obtain the proper length, offset, and biomechanics of the replaced joint.

Another object of the present invention is to provide an improved pinless surgical navigation method for use in hip and shoulder replacement surgery that produces more accurate measurements of limb length and offset by positioning femoral or humeral navigational tracking sensors on the femoral or humeral prosthesis and closer to the joint that is being replaced.

Another object of the present invention is to provide an improved pinless surgical navigation method for use in hip and shoulder replacement surgery that will accurately recreate limb length and offset by determining the ideal femoral and humeral component position from intra-operative navigational measurements of the distance between the center of rotation of the joint and the femoral and humeral prosthesis, rather than from pre-operative templating and planning.

A further object of the present invention is to provide an accurate measurement of the pre-operative and post-operative femoral or humeral limb length and femoral or humeral offset, the pre-operative and post-operative acetabular height and acetabular offset, and the pre-operative and post-operative total limb length and total offset.

A still further object of the present invention is to provide an improved pinless surgical navigation method for use in hip and shoulder replacement surgery that can be used in conjunction with known surgical procedures, such as: determining the center of rotation of the a joint through arcs of motion of the limb, through mapping the surface of the femoral head or humeral head, and can be accomplished through either a superior approach while the patient's natural femoral or humeral head is still within the patient's natural acetabulum, or by a traditional approach with the femoral or humeral head dislocated but still attached to the femoral or humeral shaft.

One aspect of the present invention relates to a method of performing a total arthroplasty of a ball and socket joint using a surgical navigation system wherein the joint has a socket and a limb having a ball shaped head at a proximal end of the limb near the socket that includes insertion of the femoral or humeral broach and/or femoral or humeral prosthesis into the femoral or humeral canal such that the distance between the broach and/or prosthesis and the center of rotation of the limb equals the available femoral or humeral prosthetic neck length and angle, plus or minus any intended limb lengthening.

Another aspect of the present invention relates to a method of performing a total arthroplasty of a ball and socket joint using a surgical navigation system which includes insertion of the femoral or humeral broach and/or femoral or humeral prosthesis into the femoral or humeral canal such that the distance between the broach and/or prosthesis and the center of rotation of the limb equals the available femoral or humeral prosthetic head sizes, plus or minus any intended limb lengthening.

Another aspect of the present invention relates to a method of performing a total arthroplasty of a ball and socket joint using a surgical navigation system which includes determining the center of rotation of the joint prior to cutting the femoral or humeral neck, which be determined with surgical navigation by rotating the femoral head in the acetabulum and calculating the center of rotational movements of the femur. The center of rotation can also be determined by surface mapping the femoral head to determine the volumetric center of the femoral head.

A further aspect of the present invention relates to a method of performing a total arthroplasty of a ball and socket joint using a surgical navigation system which includes preparing the femoral or humeral canal to receive the femoral or humeral prosthesis prior to cutting the femoral or humeral neck through either a superior approach while the patient's natural femoral or humeral head is still within the patient's natural acetabulum, or by a traditional approach with the femoral or humeral head dislocated but still attached to the femoral or humeral shaft.

A still further aspect of the present invention relates to a method of performing a total arthroplasty of a ball and socket joint, such as the hip or shoulder, using a surgical navigation system which includes: inserting the femoral or humeral broach and/or prosthesis into the femoral or humeral canal, determining the distance between the femoral or humeral broach and/or prosthesis and the center of rotation of the hip or shoulder; adjusting the position of the femoral or humeral broach and/or prosthesis to a pre-determined distance from the center of rotation of the hip or shoulder joint; and gradually increasing the size of broaches in the canal until the distance between the broach and the center of rotation of the joint is identical (plus or minus any intended changes in leg length or offset) to the available prosthetic neck length and angle.

Other objects, aspects, features and advantages of the invention will become apparent from time to time throughout the specification and claims as hereinafter related.

### BRIEF DESCRIPTION OF THE DRAWINGS

FIGS. 1A and 1B are block diagrams illustrating the major steps in the present method of performing a total arthroplasty of a ball and socket joint using a pinless surgical navigation system.

FIG. 2A is a frontal anatomical view of the pelvis, the hip joint, and the top of a femur illustrating schematically the method of determining the center of rotation of the a joint through arcs of motion of the limb.

FIG. 2B is a frontal anatomical view of the pelvis, the hip joint, and the upper portion of a femur illustrating schematically the method of determining the center of rotation of a hip joint through mapping the surface of the femoral head or humeral head.

FIG. 3A is a frontal anatomical view showing, somewhat schematically, the femoral head located in the acetabular socket in the superior approach, in preparation of inserting the femoral broach into the femoral canal.

FIG. 3B is a frontal anatomical view showing, somewhat schematically, a navigational tracking sensor attached to the implanted femoral component and the center of rotation of the hip joint determined relative to the femoral and/or pelvic navigation tracking sensors through the arcs of motion technique in the superior approach.

FIG. 3C is a frontal anatomical view illustrating, somewhat schematically, the step of determining the distance between the femoral component and the center of rotation of the hip joint using a navigation tracking sensor attached to the femoral component and an optional navigational tracking sensor attached to the pelvis, the navigational computer and a database of the implant geometry are conventional in the art, and therefore not shown.

FIG. 3D is a frontal anatomical view illustrating, somewhat schematically, the step of determining the distance between the center of the acetabular reamer and the center of rotation of the hip joint using the pelvic navigational tracking sensor as shown in FIG. 3B.

FIG. 4A is a frontal anatomical view showing, somewhat schematically, insertion of the femoral broach into the femoral canal with the femoral head still attached to the femoral shaft, but with the femoral head dislocated out of the acetabular socket in a traditional hip approach.

FIG. 4B is a frontal anatomical view showing, somewhat schematically, the femoral head placed back into the acetabulum, a navigational tracking sensor attached to the implanted femoral component, and the center of rotation of the hip joint determined relative to the femoral and/or pelvic navigation tracking sensors through the arcs of motion technique in a traditional approach.

FIG. 4C is a frontal anatomical view showing, somewhat schematically, the femoral head dislocated out of the acetabular socket in a traditional hip approach, and the step of mapping the surface geometry of the femoral head using a navigational probe to determine the volumetric center of rotation of the native femoral head.

FIG. 4D is a frontal anatomical view showing, somewhat schematically, the femoral head placed back into the acetabulum, and a first navigational tracking sensor attached to the implanted femoral component and a second navigational tracking sensor attached to the pelvic bone for determining the distance between the femoral component, the center of rotation of the hip joint, and a pelvic reference point. The center of rotation of the hip joint is also defined relative to the pelvic navigation tracking sensor.

FIG. 5A is a frontal anatomical view showing, somewhat schematically, the step of determining the distance between the implanted femoral component and a reference point on the pelvis using navigational tracking sensors attached to the femoral component and to the pelvic bone before the femoral neck is cut and before the femoral head is removed.

FIG. 5B is a frontal anatomical view showing, somewhat schematically, the step of determining the distance between the implanted femoral component and a reference point on the pelvis using navigation tracking sensors attached to the femoral component and the pelvic bone after the femoral neck has been cut, the femoral head has been removed, the acetabular socket has been prepared, and the acetabular component implanted, and trial head and neck components have been inserted.

FIG. 6 is a top plan view of the top of a femoral prosthesis illustrating, schematically, a method of calculating the femoral prosthesis malrotation as measured as the angle from the neck axis of the femoral prosthesis and the neck axis of the native femoral head and neck, as determine by the center of rotation of the hip.

FIG. 7A is a frontal anatomical view showing, somewhat schematically, a shoulder replacement with a humeral broach/prosthesis inserted into the humeral canal with the humeral head attached to the humeral shaft.

FIG. 7B is a frontal anatomical view showing, somewhat schematically, the shoulder replacement with a navigational tracking sensor attached to the implanted humeral broach/prosthesis for determining the center of rotation of the shoulder joint through the arcs of motion technique.

FIG. 7C is a frontal anatomical view showing, somewhat schematically, the shoulder replacement with a navigational tracking sensor attached to the implanted humeral broach/prosthesis and mapping the surface geometry of the humeral head using a navigational probe.

FIG. 7D is a frontal anatomical view showing, somewhat schematically, a shoulder replacement prosthesis with a similar size, shape, and center of rotation as the original shoulder joint.

### DESCRIPTION OF THE PREFERRED EMBODIMENTS

In the following discussion, the term, femoral component, refers to either the femoral broach that is used to prepare the shape of the femoral canal or the final femoral prosthesis. The term, humeral component, refers to either the humeral broach that is used to prepare the shape of the humeral canal or the final humeral prosthesis. A hip joint is described and depicted in some of the drawings, for purposes of example only, but not limited thereto, and it should be understood that present invention relates to a method of performing a total arthroplasty of a ball and socket joint, such as the hip or shoulder.

It should also be understood that the present pinless surgical navigation method can be used in conjunction with known surgical procedures, such as: determining the center of rotation of the a joint through arcs of motion of the limb, through mapping the surface of the femoral head or humeral head, and can be accomplished through either a superior approach while the patient's natural femoral or humeral head is still within the patient's natural acetabulum, or by a traditional approach with the femoral or humeral head dislocated but still attached to the femoral or humeral shaft, as will be described hereinafter.

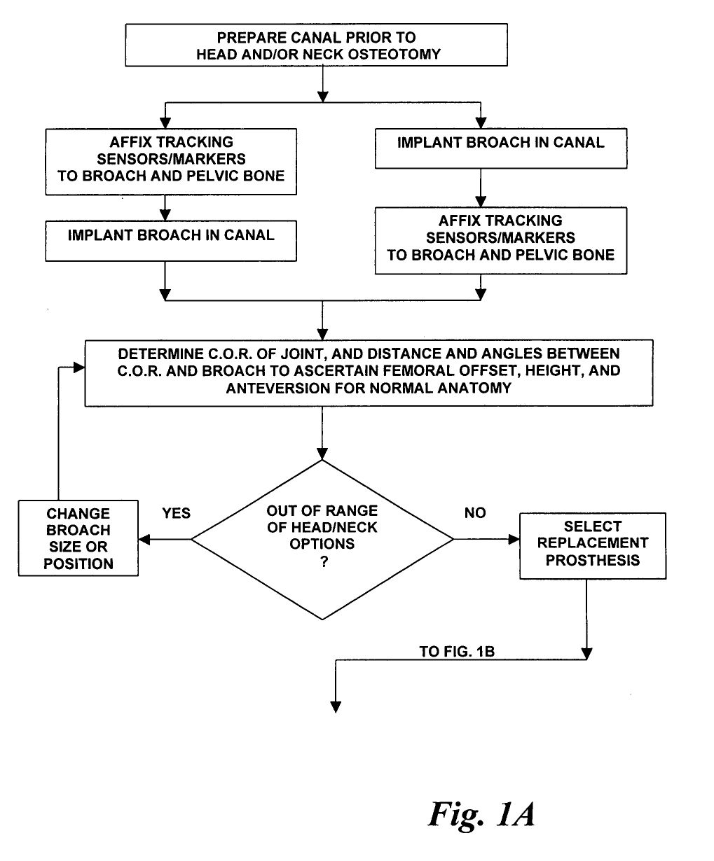

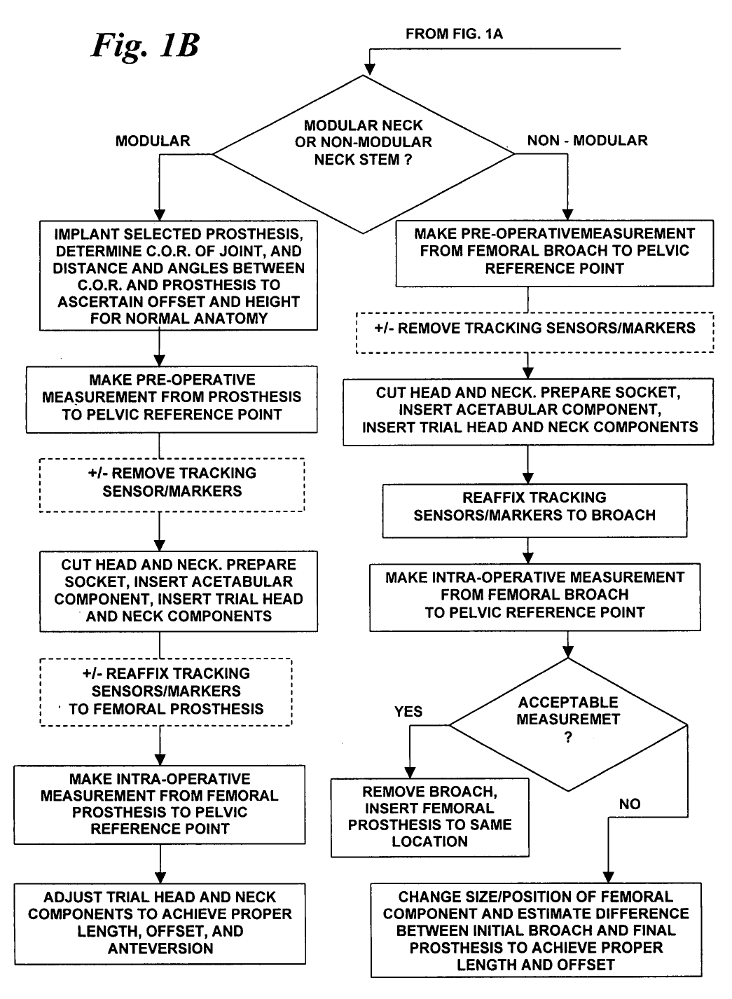

Referring now to FIGS. 1A and 1B, for purposes of an overview, and briefly stated, the major steps involved in the present method are carried out as follows:

(1) The femoral canal is prepared prior to the neck osteotomy. The femoral canal preparation can be done either with the femoral head located in the joint socket or with the head dislocated but still attached to the femoral neck and shaft. The femoral canal may be prepared by a superior approach, a posterior approach, or other traditional approach.

### (2) Navigation reference tracking sensors or markers operatively connected with a navigation processor and implant geometry database are attached to the femoral broach and optionally to the pelvic bone.

(3) Once the femoral broach is firmly positioned, the surgeon utilizes the navigation system to determine the distance between the center of rotation (COR) of the hip socket and the femoral broach. Determination of the center of rotation may be accomplished one or more of the following procedures:
(A) The center of rotation may be determined by rotating the hip in a circular motion with a navigation tracking sensor on the femoral broach and the navigation system calculates the epicenter of the rotational motion.
(B) The center of rotation may be determined by digitizing the surface of the femoral head with a navigation probe and measuring the distance from the navigation tracking sensor on the broach and the surface of the femoral head, and the navigation system calculates the center of rotation and the distance from the broach to the center of rotation.
(C) The center of rotation may be determined by biplanar radiographs/fluoroscopy.
   (4) After the distance between the center of rotation (COR) of the hip socket and the femoral broach is calculated, the surgeon would then know the femoral offset and femoral height needed to recreate the normal anatomy as a result of the calculations. The surgeon then selects the appropriately sized stem and the appropriately angled femoral neck to achieve the normal anatomy.
   (5) If the distance between the femoral broach and the center of rotation is outside of the range of options of the femoral prosthesis, the surgeon may increase or decrease the size of the femoral broach an repeat step 3 in order to obtain the correct sized femoral prosthesis.
   (6) If the distance between the center of rotation of the hip socket and the femoral broach is acceptable, the surgeon would proceed in one of the following two ways:
(A) Using a modular neck stem, the modular neck stem is inserted, and step 3 is repeated to ensure that the femoral component is still at the appropriate level. After insertion of the femoral prosthesis, a pre-operative navigated measurement to some location on the pelvic bone (i.e. the lateral acetabular rim) or a pelvic navigational tracking sensor is made. The femoral neck is cut, the socket is prepared, the acetabular component is inserted, trial head and neck components are inserted, and a repeat intra-operative navigated measurement is taken. The trial head and neck components are adjusted as necessary to achieve the desired leg length and offset.
(B) Using a non-modular stem, a preoperative navigation measurement is obtained between the femoral broach and some reference point on the pelvis using navigation tracking sensors attached to the femoral broach and the pelvic bone. The femoral broach is left in the femoral canal, the femoral neck is cut, the acetabular socket is prepared, the acetabular component is inserted, a trial head and neck are attached to the femoral broach, and the hip is reduced. The navigation tracking sensor is reattached to the femoral broach and the pelvic bone, and a repeat measurement is made between the femoral broach and some reference point on the pelvis using navigation tracking sensors attached to the femoral broach and the pelvic bone. If the repeat measurement is acceptable in terms of leg length and offset, then the femoral broach is removed and the correctly sized and neck angled femoral prosthesis is inserted to the same location as the femoral broach was. In so much as the surgeon can accurately position the femoral component to the same position as the femoral broach, the overall accuracy of this technique is preserved. If the repeat measurement is unacceptable, the surgeon could change the size or position of the femoral component and estimate the difference in position from the initial broach and the final prosthesis. The amount of change between the two positions would correspond to the error in leg length and offset.

In an alternate method, a navigation tracking sensor may be attached to the femoral bone, the center of rotation is calculated by one of the methods described above, and then a broach and/or femoral prosthesis is inserted to the desired location to recreate offset and leg length.

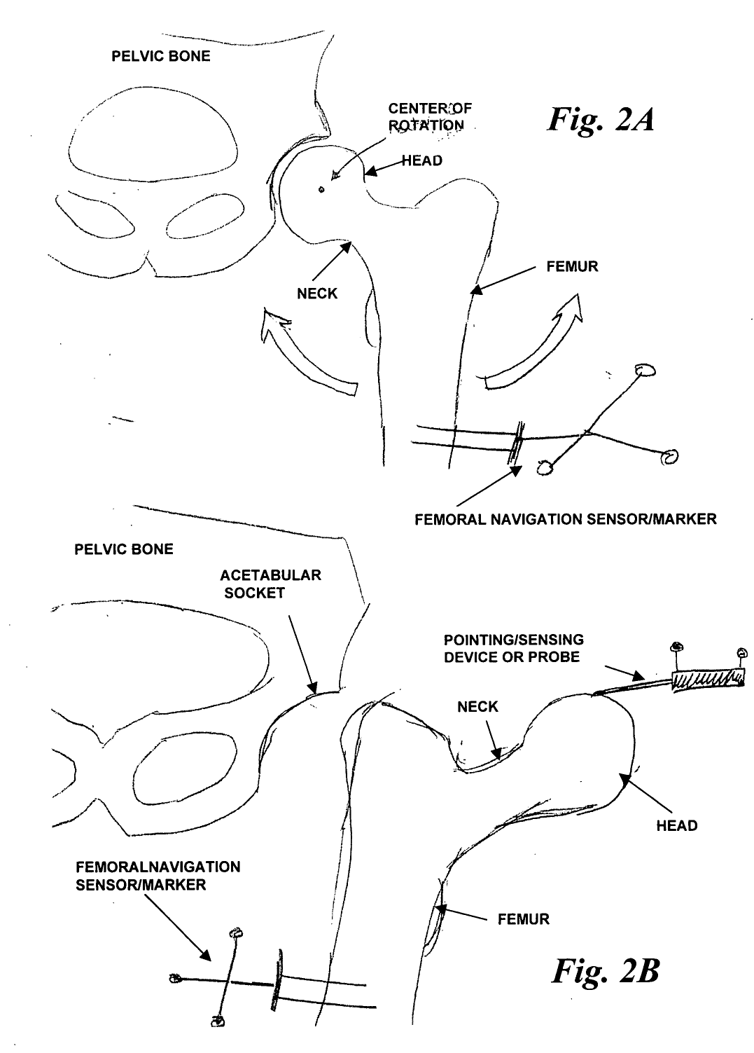

FIG. 2A illustrates, somewhat schematically, the present navigation method being used for determining the center of rotation of the hip joint through arcs of motion of the limb which is accomplished through a minimally invasive and tissue preserving surgical procedure known in the art as a superior approach while the patient's natural femoral or humeral head is still within the patient's natural acetabulum. A superior approach is disclosed in Murphy, U.S. Pat. No. 7,105,028, which is hereby incorporated herein by reference. In prior art navigation methods, a navigational tracking sensor is attached to the femur or humerus bone. In the present method, the navigational tracking sensor or marker is not attached to the bone; instead, it is attached to the prosthesis which is attached to the bone. The limb is then rotated in a circular motion and the center of rotation is measured. This technique requires that the center of rotation of the joint must not translate during the circular motion. Alternatively, a navigational tracking sensor may be attached to the pelvis bone in order to measure any movement of the pelvis during the circular motion of the limb and calculate any translational movement in the center of rotation. It should be understood that the navigational tracking sensors are operatively connected with a navigation processor and implant geometry database, which are conventional in the art, and therefore not shown.

It should also be understood that the present method may be carried out using a superior approach, a posterior approach, or other traditional approach.

FIG. 2B illustrates, somewhat schematically, the present navigation method being used for determining the center of rotation of the hip joint by mapping the surface geometry of the femoral or humeral head using a surgical navigation system wherein the navigational tracking sensor is attached to the femoral or humeral component and the surface geometry of the head is measured relative to the navigational tracking sensor using a conventional calibrated trackable pointing or sensing device, of the type known to those skilled in the art. The center of rotation can be calculated from the volumetric center of the femoral head or through measuring the arcs of the surface geometry and calculating the center of all the measured arcs.

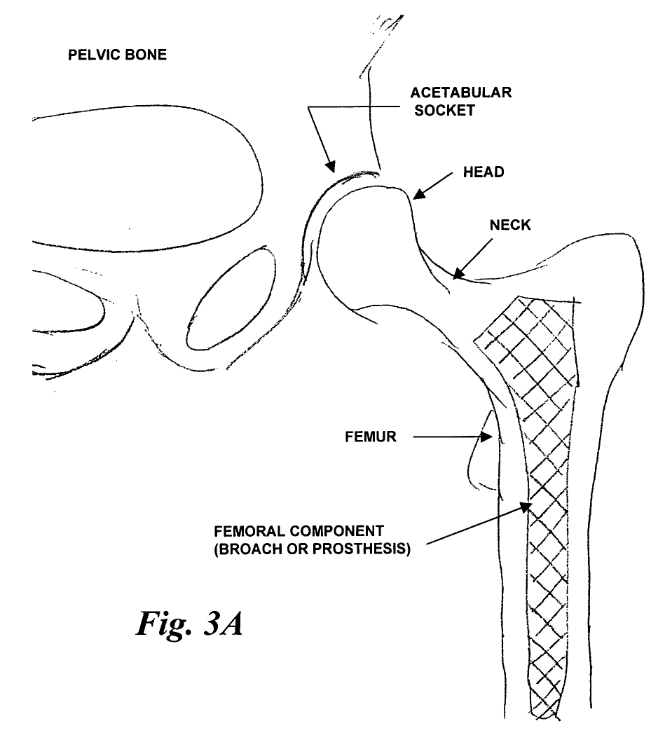

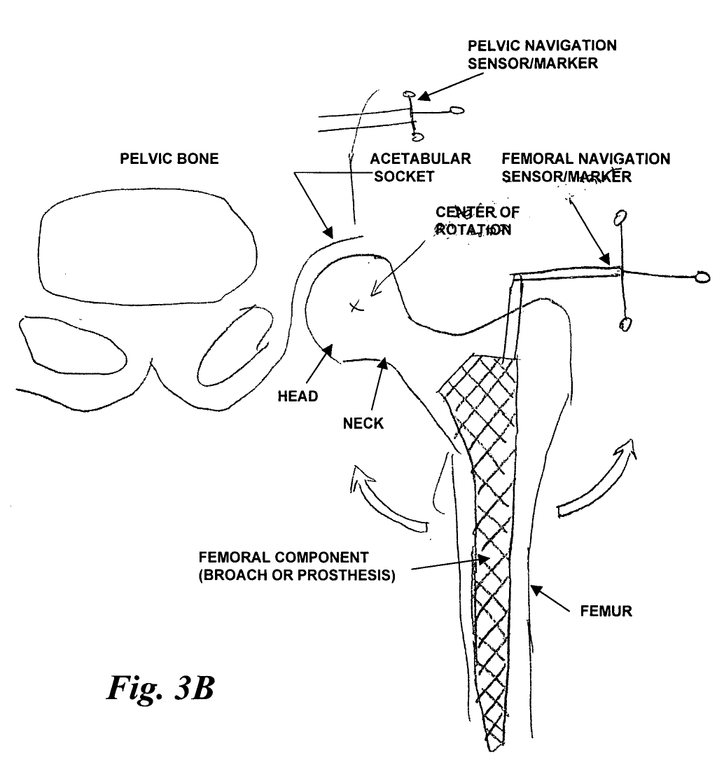

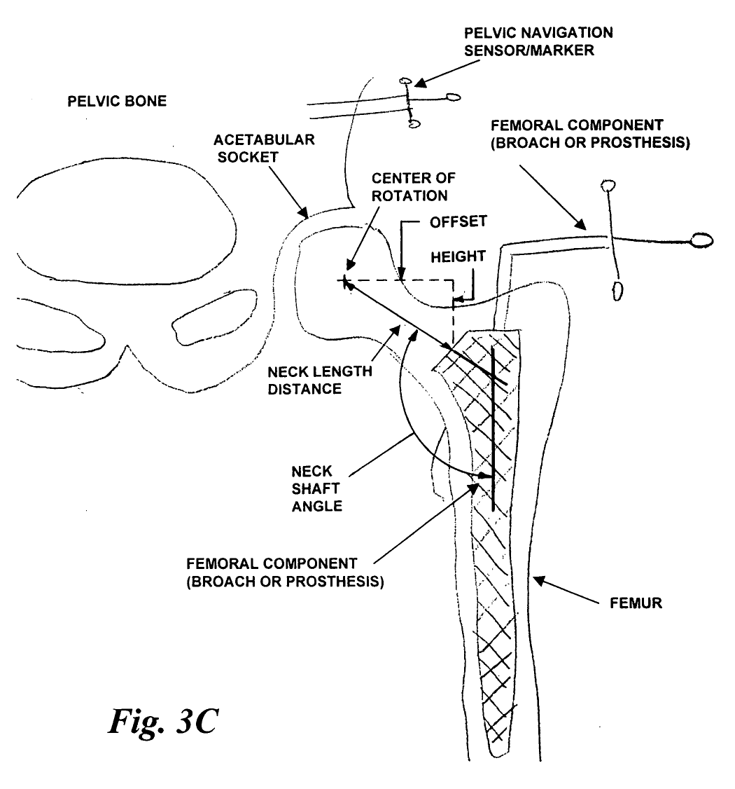

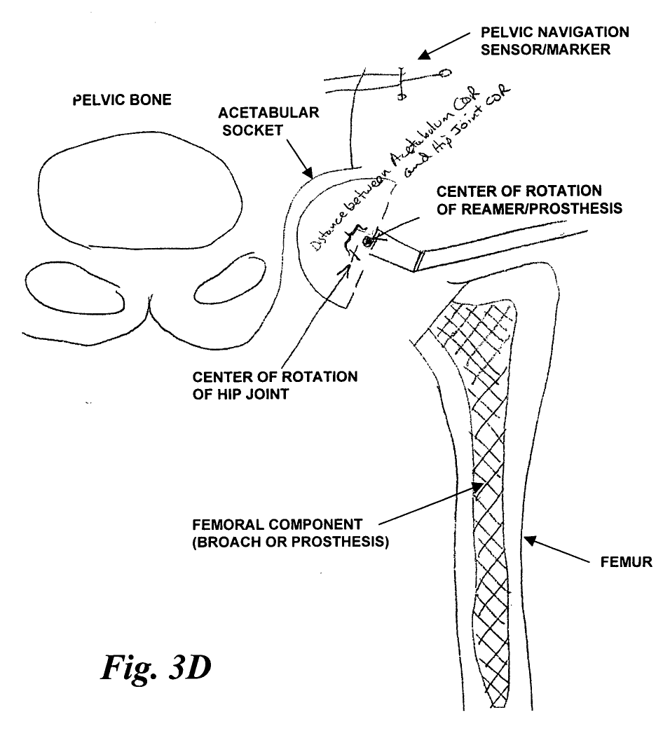

FIG. 3A illustrates, somewhat schematically, the femoral head located in the acetabular socket which may accessed by the tissue preserving superior approach disclosed in Murphy, U.S. Pat. No. 7,105,028, which is hereby incorporated herein by reference, in preparation of inserting the femoral broach into the femoral canal while the femoral head is located in the acetabular socket.

As shown somewhat schematically in FIG. 3B, a navigational tracking sensor or marker is attached to the implanted femoral component and the center of rotation is determined through the arcs of motion technique shown and described with reference to FIG. 2A and FIG. 3A. The femoral component is implanted before changing the anatomic relationship between the femoral bone and the pelvic bone, and therefore, the implanted femoral component acts as the femoral reference point instead of the femoral bone. In this method, the navigational tracking sensor or marker is attached to the thread portion of the femoral component (or similar attachment mechanism), such that the navigational tracking sensor or marker can be removed and reattached multiple times to allow the surgeon to perform the necessary surgical steps without interference from the navigational tracking sensor or marker. Alternatively, rather than attaching and removing the navigational tracking sensor or marker to and from the femoral component, a permanent navigational marker (i.e. radio frequency marker) may be fixed to the femoral component and used to make the necessary navigational measurements.

A navigational tracking sensor or marker can also be attached to the pelvic bone to allow additional measurements and more accurate positioning of the acetabular prosthesis, whereby the center of rotation of the hip joint can be determined relative to the femoral and/or pelvic navigational tracking sensor or marker so that the acetabular component can be positioned relative to the center of rotation of the joint.

As shown somewhat schematically in FIG. 3C, the distance between the femoral component and the center of rotation of the hip using the navigation tracking sensor or marker depicted in FIG. 3B and/or a navigational tracking sensor or marker attached to the pelvis. If the length and angle between the femoral component and the center of rotation does not correspond to an available neck length, neck/shaft angle, anteversion angle of prosthetic neck and head options, then the surgeon can change the location and/or angle of the implanted femoral component relative to the femoral bone by selecting either a smaller or larger femoral component, or shaping the femoral bone to accept the femoral component is a different position.

If the surgeon is using a femoral prosthesis without a modular neck, then the surgeon must firmly implant the final femoral broach, and take all necessary navigational measurements depicted in FIGS. 3B and 3C based off the location of this femoral broach and pelvic reference point. The surgeon would then cut the femoral neck, remove the femoral head, prepare the acetabular socket, implant the acetabular component, attach the prosthetic neck and head, reduce the hip, and repeat the navigational measurements. If the measurements were acceptable, the surgeon would then implant the femoral prosthesis to the same location as the final femoral broach. The surgeon would still benefit from the measurements depicted in FIG. 3C, but the overall accuracy of this technique would depend on positioning the femoral prosthesis in the same location as the femoral broach. If the measurements were unacceptable, changes to the femoral prosthesis position could be made accordingly, but the surgeon would introduce an estimate of the difference (error) into the navigational measurements based on the estimated change between the initial and final femoral component positions.

If the surgeon is using a femoral prosthesis with a modular neck, then the surgeon can take preliminary navigational measurements with the femoral broaches, implant the femoral prosthesis to its permanent position in the femoral bone, and take additional navigational measurements based off the permanent position of the femoral prosthesis. After the additional navigational measurements based off the permanent position of the femoral prosthesis are taken, the femoral neck is cut, the femoral head is removed, the acetabular socket is prepared and the acetabular component is inserted. By using the permanent position of the femoral prosthesis with a modular neck instead of the femoral broach, the surgeon could eliminate the possible error of positioning the femoral component in a different location than the final femoral broach.

As shown somewhat schematically in FIG. 3D, the distance between the center of the acetabular reamer and the center of rotation of the hip using the pelvic navigational tracking sensor or marker discussed above with reference FIG. 3B is determined. The surgeon would know from the calculations of the distance between the femoral component and the center of rotation of the hip as discussed with reference to FIG. 3C whether he/she would be able to increase the femoral offset and femoral length to compensate for any intended changes in the acetabular component position. The surgeon could then ream the acetabular socket such that the center of the acetabular reamer corresponds to the center of rotation of the hip joint plus or minus any intended change in acetabular offset and height. The surgeon would then place the acetabular component such that the center of the acetabular component corresponds to the center of rotation of the hip joint plus or minus any intended change in acetabular offset and height. A navigational sensor or marker may be attached to the acetabular component during insertion of the acetabular component to ensure the acetabular component is implanted at the proper distance from the center of rotation of the hip joint.

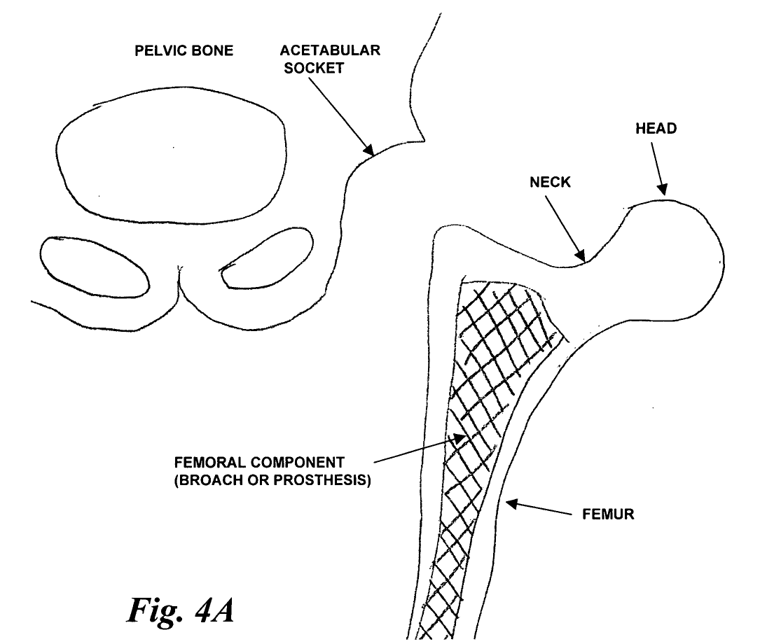

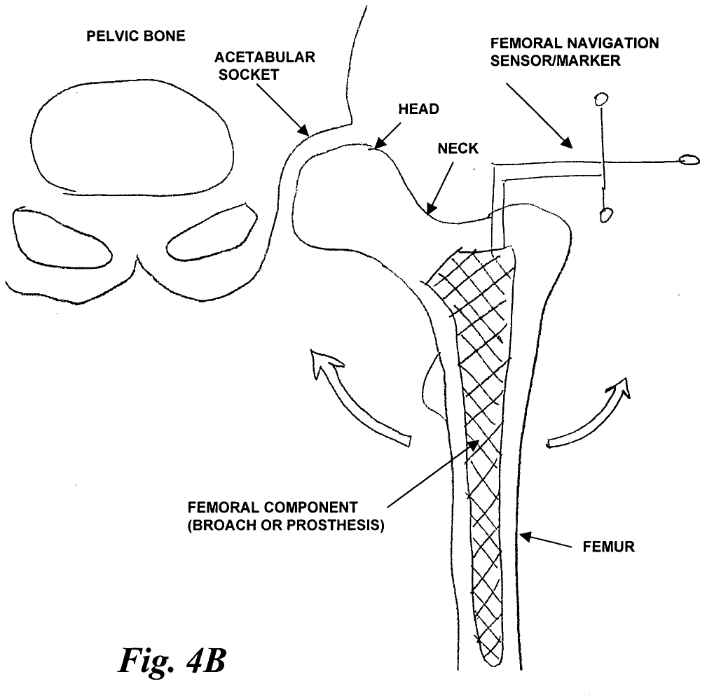

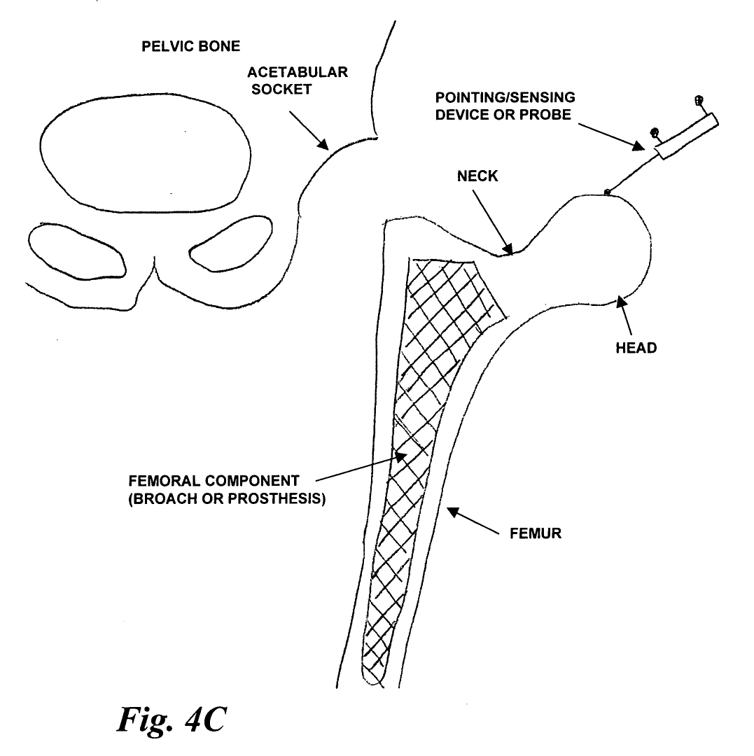

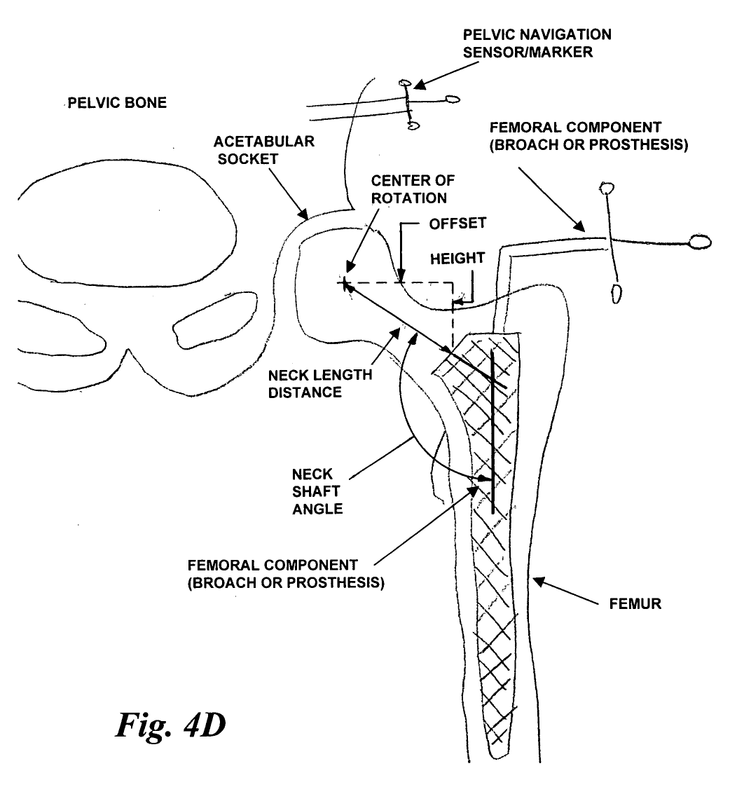

Referring now to FIG. 4A, there is shown, somewhat schematically, a method of inserting the femoral broach into the femoral canal with the femoral head H still attached to the femoral shaft, but with the femoral head dislocated out of the acetabular socket in a traditional approach. In order to accomplish this procedure, the surgeon would first dissect some of the muscle and capsule off the proximal femur and then dislocate the femoral head out of the socket. The femoral canal would then be entered with a drill placed in the superior area of the femoral neck/metaphyseal junction (i.e. piriformis fossa). Sequentially larger reamers would be inserted down the femoral canal to enlarge the femoral canal. A portion of the femoral metaphyseal bone would be removed to allow the insertion of sequentially larger femoral broaches. The final broach would be the broach that had a length from the femoral broach to the center of rotation of the hip joint that matched the available prosthetic neck length.

As shown somewhat schematically in FIG. 4B, a navigational tracking sensor or marker is attached to the implanted femoral component, the femoral head is reduced back into the acetabulum, and the center of rotation is determined through the technique shown and described above with reference to FIG. 2A and FIG. 4A. The femoral component is implanted before changing the anatomic relationship between the femoral bone and the pelvic bone, and therefore, the implanted femoral component acts as the femoral reference point instead of the femoral bone. In this method, the navigational tracking sensor or marker is attached to the thread portion of the femoral component (or similar attachment mechanism), such that the navigational tracking sensor or marker can be removed and reattached multiple times to allow the surgeon to perform the necessary surgical steps without interference from the navigational tracking sensor or marker. Alternatively, rather than attaching and removing the navigational tracking sensor or marker to and from the femoral component, a permanent navigational marker (i.e. radio frequency marker) may be fixed to the femoral component and used to make the necessary navigational measurements.

A navigational tracking sensor or marker can also be attached to the pelvic bone to allow additional measurements and more accurate positioning of the acetabular prosthesis, whereby the center of rotation of the hip joint can be determined relative to the pelvic navigational tracking sensor or marker so that the acetabular component can be positioned relative to the center of rotation of the joint. A navigational tracking sensor or marker attached to the pelvis is not necessary for the femoral component positioning, but is necessary for the acetabular component positioning relative to the center of rotation of the joint.

FIG. 4C illustrates, somewhat schematically, the present navigation method being used for determining the center of rotation of the hip joint by mapping the surface geometry of the femoral or humeral head using a navigational probe and a navigational tracking sensor or marker attached to the femoral or humeral component, as described above with reference to FIG. 2B. In this example, the femoral head is dislocated but still attached to the femoral shaft. The center of rotation can be calculated from the volumetric center of the native femoral head or through measuring the arcs of the surface geometry and calculating the center of all the measured arcs. The femoral head could then be reduced and the calculated center of rotation of the hip joint could be determined relative to the navigational tracking sensor or marker to assist with acetabular component positioning.

FIG. 4D illustrates, somewhat schematically, the method for determining the distance between the femoral prosthesis/broach and the center of rotation of the hip using the techniques discussed above with reference to FIGS. 4B and 4C. If the neck length, neck/shaft angle and anteversion angle between the femoral component and the center of rotation of the hip joint does not correspond to an available length and angle of prosthetic neck and head options, then the surgeon can change the location of the implanted femoral component relative to the femoral bone by selecting either a smaller or larger femoral component and/or shaping the femoral bone to accept the femoral component in a different position. Because the navigational tracking sensor or marker attaches to the thread portion of the femoral component (or similar attachment mechanism), the navigational tracking sensor or marker can be removed and reattached multiple times to allow the surgeon to perform the necessary surgical steps through a smaller incision. Referring again to FIG. 3D, the distance between the center of the acetabular reamer/component and the center of rotation of the hip joint is determined by using the techniques and a pelvic navigational tracking sensor or marker discussed above with reference to FIG. 4B and/or FIG. 4C. The surgeon could alter the intended acetabular component position based on the femoral component position and the amount of offset and leg length available through the femoral prosthesis.

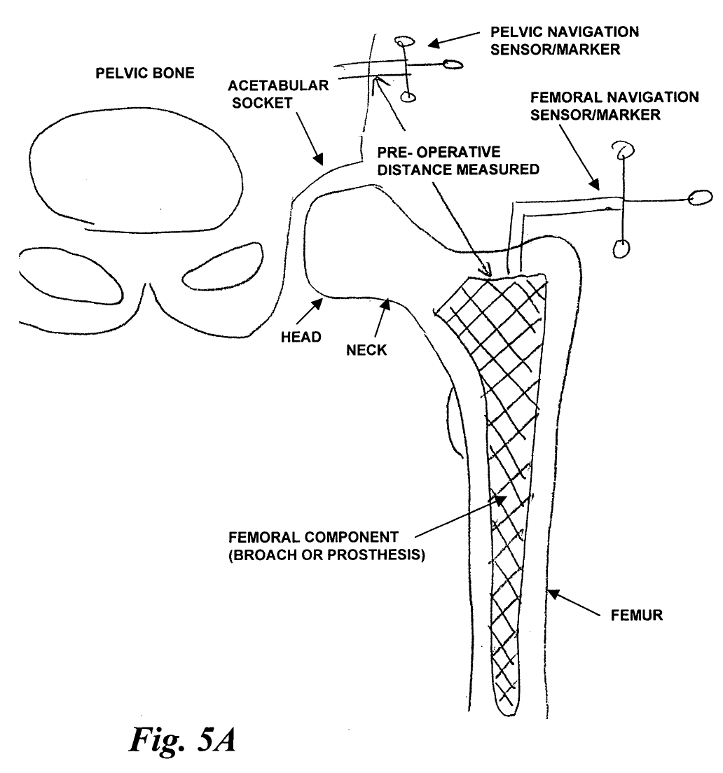

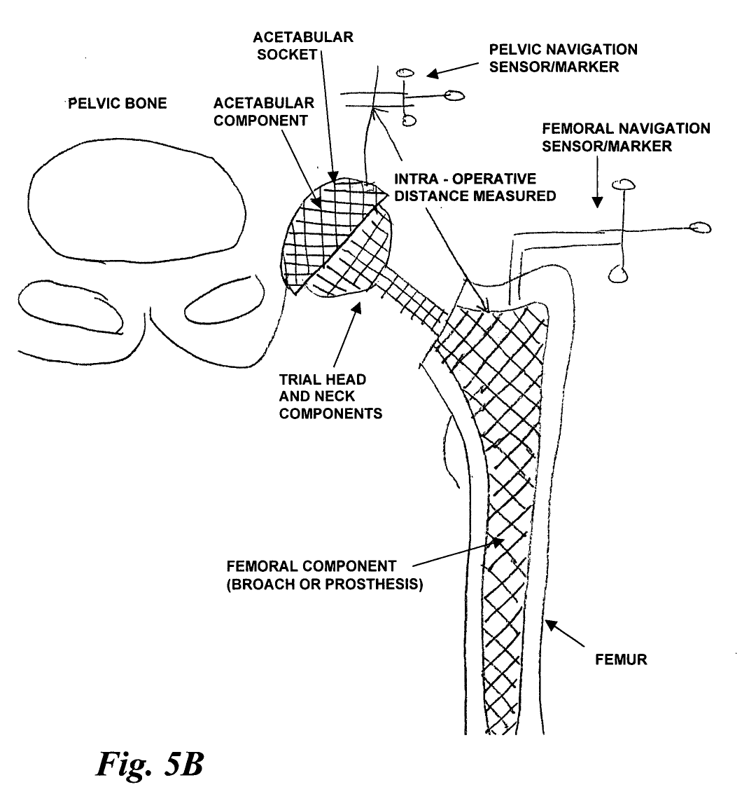

Referring now FIG. 5A there is shown, somewhat schematically, the present navigation method being used for determining the distance between the implanted femoral component and a reference point on the pelvis using a navigational tracking sensor or marker on the femoral component and a navigational tracking sensor or marker on the pelvis before the femoral neck is cut and before the femoral head is removed, which is accomplished utilizing a measurement procedure and device which is disclosed in my copending U.S. patent application Ser. No. 11/640,141, which the present application is a C-I-P of, and which is expressly incorporated herein by reference in its entirety.

The device disclosed in my copending application Ser. No. 11/640,141, is a joint/leg measurement device for determining length displacement and lateral offset displacement of a patient's femur during hip replacement surgery, and includes a reference member, a marking device, and a measurement tool. For a hip replacement, a femoral prosthetic component is inserted prior to the cutting of the femoral neck. As a result, the prosthesis preferably is placed into the canal prior to the disruption of the natural anatomical relationship of the hip joint and the ilium. The reference member is attachable to a femoral prosthetic component thus allowing the prosthesis in combination with the reference member to act as a reference for the leg measurement tool. The reference member thus provides a stable component which extends proximately along the femoral axis. In this manner, a leg measurement device can provide leg length and lateral offset displacement measurements by measuring the relative movement of the femoral axis with reference to the ilium reference location. The reference member is transversely intersected by the leg measurement tool, which is placed at the ilium reference location thereby permitting the physician to keep track of the relative movement of the femur both before and after hip replacement surgery. As such, by providing this reference member attached to the femoral prosthetic component, the reference member can continually keep track of the femoral axis thereby providing more reliable measurements of displacements in the femur relative to the ilium.

Thus, the femoral component itself acts as an extension of the femoral axis to track changes in the leg length and lateral offset displacements. The leg measurements are more accurate because the measurements are taken closer to the joint without bending pins, which leads to inaccuracy. The leg measurement tool is preferably always pointing in the same reference direction when placed at the ilium reference location. This permits the surgeon to measure the movements and rotations of the femur relative to the ilium. As a result, the joint can always be placed in the same position between measurements.

The device may also have a guiding member that inserts through an aperture in the reference member. This guiding member is utilized to mark the ilium reference location on the ilium. In order to mark this reference location, the guiding member can be inserted through the reference member for inserting a guide pin through the passage in the guiding member and into the ilium. The ilium is then drilled and a marking apparatus, which may be a cannulated screw, is then inserted into the ilium reference location. The leg measurement device can be inserted into the marking apparatus repetitively. Thus, by placing the leg measurement tool into the marking apparatus at the ilium reference location, the leg measurement tool maintains a reproducible reference line from the ilium reference location to the reference member. When the leg is repositioned such that the reference member is perpendicular to the measurement tool, the leg measurement tool will preferably always maintain the same axis relative to the marking apparatus and will preferably always be perpendicular to the femoral axis thereby assuring that the leg is repositioned in the original rotation.

FIG. 5B shows, somewhat schematically, the present navigation method being used for determining the distance between the implanted femoral component and a reference point on the pelvis using a navigational tracking sensor or marker attached to the femoral component and a navigational tracking sensor or marker attached on the pelvis after the femoral neck has been cut, the femoral head has been removed, the acetabular socket has been prepared, and the acetabular component and trial head and neck components have been inserted. The surgeon can then determine the distance between the implanted femoral component and a navigational tracking sensor or marker with either trial modular neck and trial modular head components implanted in the hip joint or with final modular neck and final modular head components implanted in the hip joint, which may be accomplished utilizing the procedures disclosed in my copending U.S. patent application Ser. No. 11/640,141.

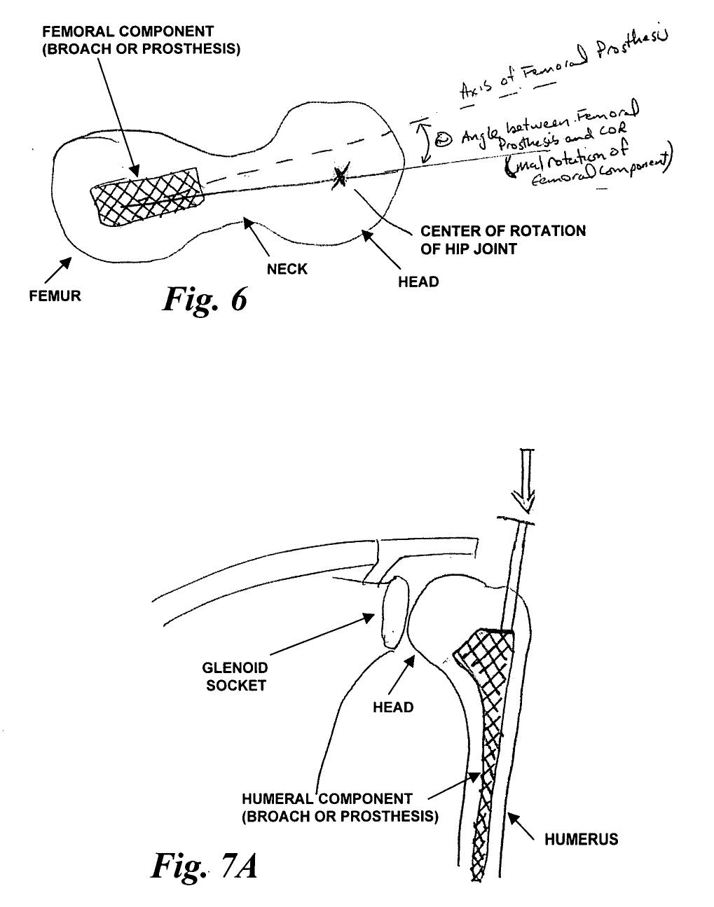

FIG. 6 is a top plan view of the top of a femoral prosthesis illustrating, schematically, a method of calculating the femoral prosthesis malrotation as measured as the angle between the neck axis of the femoral prosthesis (femoral prosthesis anteversion) and the native femoral anteversion, which is determined by the center of rotation of the hip joint and the axis of the femoral canal. Alternatively, the native femoral anteversion could also be determined from mapping the surface geometry of the femoral head as shown in FIGS. 2b and 4c. If the surgeon has implanted the femoral component in a slightly different rotation compared to the native anatomy, then the neck axis of the femoral prosthesis will not coincide with the neck axis of the native femoral head and neck. The surgeon would then have the opportunity to either change the rotation of the femoral component or select an anteverted modular femoral prosthetic neck to compensate for the malrotation.

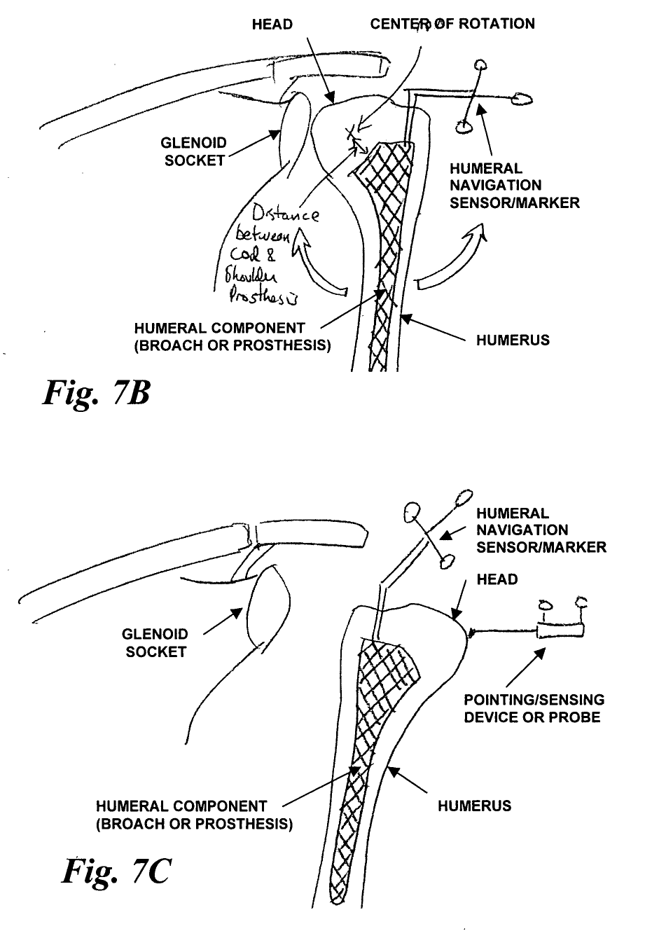

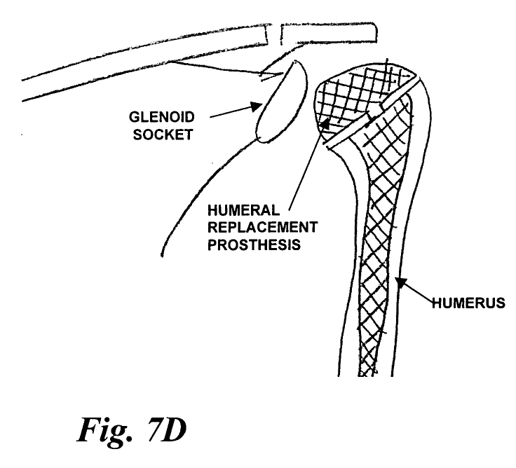

Referring now to FIG. 7A, there is shown, somewhat schematically, a shoulder replacement with the humeral component inserted into the humeral canal with the humeral head still attached to the humeral shaft. The humeral head could be located either in the glenoid socket of the scapula, or dislocated outside of the socket. The surgeon inserts a drill just medial to the greater tuberosity, gradually enlarge the humeral canal with sequentially larger reamers, remove a small amount of metaphyseal bone, and then inserts sequentially larger broaches until the distance from the broach to the surface of the humeral head matches the available modular humeral prosthetic head sizes. The surgeon would also have the opportunity to change the anteversion angle relative to the native humeral anteversion as described in FIG. 6.

FIG. 7B shows, somewhat schematically, the shoulder replacement with a navigational tracking sensor or marker attached to the implanted humeral component whereby determination of the center of rotation of the shoulder joint is carried out by the procedure described above with reference to FIG. 2A. Gentle rotational movement of the humerus is performed and the center of the rotation is calculated relative to the navigational tracking sensor or marker. Because the navigational tracking sensor or marker attaches to the thread portion of the humeral component (or similar attachment mechanism), the navigational tracking sensor or marker can be removed and reattached multiple times to allow the surgeon to perform the necessary surgical steps without interference from the navigational tracking sensor or marker. Alternatively, rather than attaching and removing the navigational tracking sensor or marker to and from the humeral component, a permanent navigational marker (i.e. radio frequency marker) may be permanently fixed to the humeral component and used to make the necessary navigational measurements. The humeral prosthetic head size and offset is selected from the available modular head sizes to recreate the same center of rotation of the shoulder joint.

FIG. 7C illustrates, somewhat schematically, the shoulder replacement with a navigational tracking sensor or marker attached to the implanted humeral prosthesis and mapping the surface geometry of the humeral head using a navigational probe, as described above with reference to FIGS. 2B and 4C. The navigational system calculates the size, shape, and location of the native humeral head relative to the position of the humeral component and assists the surgeon in determining the appropriate size, shape, and location of the prosthetic humeral head. FIG. 7D shows, somewhat schematically, a shoulder replacement prosthesis with a similar size, shape and center of rotation as the original shoulder joint.

While this invention has been described fully and completely with special emphasis upon preferred embodiments, it should be understood that within the scope of the appended claims the invention may be practiced otherwise than as specifically described herein. The foregoing disclosure and description of the invention is illustrative and explanatory thereof. No limitations are intended to the details of construction or design, herein shown, or to the methods described herein, other than is described in the claims below.

1. A method of performing a total arthroplasty of a ball and socket joint of a patient using a surgical navigation system wherein the joint has a socket formed of bone and a limb formed of bone, the limb having a ball shaped head and neck at a proximal end near the socket, the method comprising the steps of:
affixing a surgical navigation tracking sensor or marker operatively connected with a navigation processor and implant geometry database to the limb bone and/or to an implanted limb component selected from the group consisting of a broach and a prosthesis;
determining the center of rotation of the ball and socket joint relative to the navigation sensor or marker affixed to the limb bone and/or the implanted limb component; and
implanting either of a traditional stemmed limb component or a socket resurfacing limb component to a known distance from the center of rotation of the ball and socket joint.

2. The method according to claim 1, wherein
said step of affixing comprises affixing said surgical navigation tracking sensor or marker to said implanted limb component such that the surgical navigation tracking sensor or marker is not attached directly to any portion of the limb bone.

3. The method according to claim 1, wherein
said step of affixing comprises affixing said surgical navigation tracking sensor or marker to said implanted limb component prior to changing the anatomic relationship between the limb and socket.

4. The method according to claim 1, wherein
said step of affixing comprises affixing said surgical navigation tracking sensor or marker to said limb component prior to implanting said limb component in said limb.

5. The method according to claim 1, wherein
said step of affixing comprises affixing said surgical navigation tracking sensor or marker after said limb component is implanted and prior to osteotomy and removal of said head and/or neck.

6. The method according to claim 1, wherein
said step of affixing comprises affixing said surgical navigation tracking sensor or marker to said implanted limb component while the head of said limb is disposed within said socket and attached to said limb using a minimally invasive surgical procedure known as the superior approach, including the step of making a superior incision for accessing said ball and socket joint.

7. The method according to claim 1, wherein
said step of affixing comprises affixing said surgical navigation tracking sensor or marker to said implanted limb component while the head of said limb is reduced, dislocated or subluxed from said socket and attached to said limb.

8. The method according to claim 7, comprising the further step of:
replacing said head back into said socket with said surgical navigation tracking sensor or marker affixed to said implanted limb component for calculating the center of rotation of said joint.

9. The method according to claim 1, wherein
said step of implanting said limb component comprises implanting said limb component into the proximal end of said limb while the head of said limb is reduced, dislocated or subluxed from said socket and prior to osteotomy and removal of said head and/or neck.

10. The method according to claim 1, comprising the further steps of:
removing said surgical navigation tracking sensor or marker from said limb component to facilitate performing an operative procedure; and
thereafter reaffixing said surgical navigation tracking sensor or marker to said limb component in the same position as before for acquiring additional accurate navigational measurements.

11. The method according to claim 1, wherein
said ball and socket joint is a hip joint, said socket is an acetabular socket on the pelvis, said limb is a femur having a femoral shaft and canal, said head is a femoral head adjoined to said proximal end by said neck, said implanted limb component is a femoral broach or femoral prosthesis implanted in the femoral canal, and said surgical navigation tracking sensor or marker is affixed to the implanted femoral broach or femoral prosthesis.

12. The method according to claim 1, wherein
said ball and socket joint is a shoulder joint having a scapula with a glenoid socket, said limb is a humerus having a humeral shaft and canal, said head is a humeral head adjoined to said proximal end, and said implanted limb component is a humeral broach or humeral prosthesis implanted in the humeral canal, and said surgical navigation tracking sensor or marker is affixed to the implanted humeral broach or humeral prosthesis.

13. A method for accurately positioning a replacement prosthesis during a total hip arthroplasty of a patient's hip joint using a pinless surgical navigation procedure, the hip joint having a pelvis with an acetabular socket and a native femoral head and neck near the socket adjoined by a neck to a proximal end of a femur bone having a femoral shaft and a femoral canal, the method comprising the steps of:
preparing the femoral canal to receive a femoral broach or femoral prosthesis component prior to osteotomy of the native femoral head and/or neck;
affixing a surgical navigation tracking sensor or marker operatively connected with a navigation processor and implant geometry database to the femoral broach or femoral prosthesis component prior to changing the anatomic relationship between the femur and socket such that the surgical navigation tracking sensor or marker is not attached directly to any portion of the femur bone;
affixing a second surgical navigation tracking sensor or marker operatively connected with the navigation processor and implant geometry database to the pelvic bone;
implanting the femoral broach or femoral prosthesis component in said femoral canal at the proximal end of the femur;
determining the center of rotation of the hip joint and utilizing the surgical navigation tracking sensor or marker and navigation processor to calculate the distance and angles between the center of rotation of the joint and a point on the femoral broach or femoral prosthesis component to ascertain the femoral offset and height needed to recreate the patient's normal anatomy;
selecting a replacement prosthesis having a femoral head and neck with size, angle, length, and anteversion corresponding to the calculated distances and angles between the femoral broach or femoral prosthesis component and the center of rotation to achieve the patient's normal anatomy; and
if the distance between the femoral broach or femoral prosthesis component and the center of rotation is outside of the range of options of the replacement femoral prosthesis, increasing or decreasing the size of the femoral broach or femoral prosthesis component and/or reshaping the femoral bone to accept the femoral component in a different position and repeating the steps of determining the center of rotation of the hip joint as needed to obtain an available correct sized replacement femoral prosthesis.

14. The method according to claim 13, wherein
said step of determining the center of rotation of said joint comprises rotating the femur in a circular motion and utilizing the surgical navigation tracking sensor or marker and navigation processor to calculate the center of rotation.

15. The method according to claim 13, wherein
said step of determining the center of rotation of said joint comprises digitally mapping the surface geometry of the femoral head utilizing a navigation probe and the navigation processor.

16. The method according to claim 13, wherein
said step of implanting the femoral broach or femoral prosthetic component includes inserting the femoral broach or femoral prosthetic component a known distance from the center of rotation of the hip joint.

17. The method according to claim 13, comprising the further step of:
adjusting the size and position of said femoral broach or femoral prosthesis component as necessary based on the calculated distance between the center of rotation and the femoral broach or femoral prosthesis component and known lengths and angles of available replacement femoral prosthetic necks.

18. The method according to claim 13, comprising the further step of:
determining the degrees of malrotation of the femoral broach or femoral prosthetic component by calculating the malrotation angle between the neck axis of the femoral broach or femoral prosthesis component and the neck axis of the native femoral head and neck as determined by the center of rotation of the hip joint and the center of the femoral canal.

19. The method according to claim 13, comprising the further step of:
determining the distance between the femoral broach or femoral prosthesis component and the articular surface of the femoral head.

20. The method according to claim 19, comprising the further step of:
adjusting the size and position of the femoral broach or femoral prosthesis component based on the calculated distance between the articular surface of the femoral head and the femoral prosthesis component and known lengths and angles of available replacement femoral prosthetic necks to adjust the patient's leg length and offset.

21. The method according to claim 13, comprising the further step of:
affixing said navigation tracking sensor or marker to the femoral broach or prosthesis to measure the distance between said surgical navigation tracking sensor or marker and a fixed reference point on the pelvis prior to osteotomy of the native femoral head and/or neck to determine a pre-operative leg length and offset measurement.

22. The method according to claim 13, comprising the further step of:
affixing said navigation tracking sensor or marker to the femoral broach or prosthesis to measure the distance between said surgical navigation tracking sensor or marker and a fixed reference point on the pelvis after osteotomy of the native femoral head and/or neck to determine a post-operative leg length and offset measurement.

23. The method according to claim 13, comprising the further steps of:
affixing a second surgical navigation tracking sensor or marker to the pelvis and determining the center of rotation of the hip joint through the first said navigational tracking sensor attached to the implanted femoral broach or femoral prosthesis component and relating that location to said second surgical navigation tracking sensor or marker attached to the pelvis.

24. The method according to claim 23, comprising the further steps of:
positioning the center of rotation of an acetabular reamer relative to the acetabular socket at a distance corresponding to the determined center of rotation of the hip joint;
adjusting the acetabular reamer depth and height and position relative to the center of rotation of the joint according to the calculated femoral offset and femoral height measurements and to any possible increase in femoral offset and height with different prosthetic head and neck selections; and
reaming the acetabular socket as needed to recreate the patient's normal anatomy; and attaching a navigational marker to the acetabular component during the insertion of the acetabular component to ensure the acetabular component is implanted at the proper distance from the center of rotation of the hip joint.

25. The method according to claim 13, comprising the further steps of:
inserting the stem of a femoral prosthesis having a modular neck stem into the femoral canal;
repeating the steps of determining the center of rotation of the hip joint to ensure that the femoral component is still at the appropriate position;
making a pre-operative navigated measurement to a reference point on the pelvic bone or a pelvic navigational tracking sensor;
cutting the femoral neck;
preparing the acetabular socket and inserting the acetabular component;
inserting trial head and neck components;
making an intra-operative navigated measurement to some location on the pelvic bone or a pelvic navigational tracking sensor; and
adjusting the trial head and neck components as necessary to achieve the desired leg length and offset.

26. The method according to claim 13, comprising the further steps of:
making a preoperative navigation measurement between the femoral broach and a reference point on the pelvis using navigation trackers attached to the femoral broach and to the pelvic bone;
with the femoral broach still in the femoral canal, cutting the femoral neck;
preparing the acetabular socket;
inserting an acetabular component;
attaching a trial head and neck to the femoral broach;
reducing the hip;
reattaching a navigation tracking sensor to the femoral broach and to the pelvic bone;
making a repeat measurement between the femoral broach and reference point on the pelvis; and
upon obtaining an acceptable repeat measurement in terms of leg length and offset, removing the femoral broach and inserting a correctly sized and neck angled femoral prosthesis to the same location as the femoral broach was located; or
upon obtaining an unacceptable repeat measurement in terms of leg length and offset, changing the size and/or position of the femoral component and estimating the difference in position from the initial broach and the final prosthesis, and utilizing the estimated amount of change between the two positions to achieve the desired leg length and offset.

27. A method for accurately positioning a replacement prosthesis during a shoulder arthroplasty of a patient's shoulder joint using a pinless surgical navigation procedure, the shoulder joint having a scapula with a glenoid socket and a native humeral head near the glenoid socket adjoined by a neck to a proximal end of a humerus bone having a humeral shaft and a humeral canal, the method comprising the steps of:
preparing the humeral canal to receive a humeral broach or humeral prosthesis component prior to osteotomy of the native humeral head and/or neck;
affixing a surgical navigation tracking sensor or marker to the implanted or soon to be implanted humeral broach or humeral prosthesis component prior to changing the anatomic relationship between the humerus and scapula such that the surgical navigation tracking sensor or marker is not attached directly to any portion of the humerus bone;
implanting the humeral broach or humeral prosthesis component in said humeral canal at the proximal end of the humerus;
determining the center of rotation of the shoulder joint and utilizing the surgical navigation tracking sensor or marker to calculate the distance and angles between the center of rotation of the joint and a point on the humeral broach or humeral prosthesis component to ascertain the humerus offset and height needed to recreate the patient's normal anatomy;
determining the surface geometry of the articular surface of the humeral head and utilizing the surgical navigation tracking sensor or marker attached to the humeral component to calculate the distance and angles between the articular surface of the humeral head and the humeral broach or humeral prosthesis to ascertain the prosthetic humeral head size and length needed to recreate the patient's normal anatomy;
selecting a replacement prosthesis having a humeral head size, angle, length, and anteversion corresponding to the calculated distances and angles between the humeral broach or humeral prosthesis component, the center of rotation, and articular surface of the humeral head to achieve the patient's normal anatomy; and
if the distance between the humeral broach or humeral prosthesis component, the center of rotation, and the articular surface of the humeral head is outside of the range of options of the replacement humeral prosthesis, increasing or decreasing the size of the humeral broach or humeral prosthesis component and repeating the steps of determining the center of rotation and articular surface of the shoulder joint as needed to obtain an available correct sized replacement humeral prosthesis.

28. The method according to claim 27, wherein
said step of determining the center of rotation of said joint comprises rotating the humerus in a circular motion and utilizing the surgical navigation tracking sensor or marker to calculate the center of rotation.

29. The method according to claim 27, wherein
said step of determining the articular surface of said joint comprises digitally mapping the surface geometry of the humeral head relative to the surgical navigational tracking sensor attached to the implanted humeral broach or humeral prosthesis in order to accurately size and position the prosthetic humeral head such that replaced humeral head has a similar size, shape and angle as the native humeral head.

30. The method according to claim 27, wherein
said step of implanting the humeral broach or humeral prosthetic component includes inserting the humeral broach or humeral prosthetic component a known distance from the center of rotation of the shoulder joint or known distance from the articular surface of the humeral head.

31. The method according to claim 30, comprising the further step of:
determining the angle between the neck axis of the implanted humeral broach or humeral prosthesis and the neck axis of the native humeral head and the amount of anteversion or retroversion of the humeral broach or humeral prosthesis; and
adjusting the rotation of the humeral broach or humeral prosthesis, or selecting an offset humeral head to achieve the patient's normal anatomy.
